SEQUENCE LISTING (16 sequences)

SEQ ID NO: 1 - DNA, 24 bp, Artificial Sequence
  Forward primer for GAPDH
gtatgacaacgaatttggctacag

SEQ ID NO: 2 - DNA, 24 bp, Artificial Sequence
  Reverse primer for GAPDH
tctctctcttcctcttgtgctctt

SEQ ID NO: 3 - DNA, 20 bp, Artificial Sequence
  Forward primer for CCR1
gaaacatcctggtggtcctg

SEQ ID NO: 4 - DNA, 20 bp, Artificial Sequence
  Reverse primer for CCR1
aagagcaggtcagaaatggc

SEQ ID NO: 5 - DNA, 20 bp, Artificial Sequence
  Forward primer for CCR2
agctgaagtgcttgactgac

SEQ ID NO: 6 - DNA, 20 bp, Artificial Sequence
  Reverse primer for CCR2
ttgcattcccaaagacccac

SEQ ID NO: 7 - DNA, 20 bp, Artificial Sequence
  Forward primer for CCR3
gggcagatacatcccattcc

SEQ ID NO: 8 - DNA, 20 bp, Artificial Sequence
  Reverse primer for CCR3
acacaatagagagttccggc

SEQ ID NO: 9 - DNA, 20 bp, Artificial Sequence
  Forward primer for CXCR4
aaatcttcctgcccaccatc

SEQ ID NO: 10 - DNA, 20 bp, Artificial Sequence
  Reverse primer for CXCR4
acttgtccgtcatgcttctc

SEQ ID NO: 11 - DNA, 20 bp, Artificial Sequence
  Forward primer for ICAM
ggagcttcgtgtcctgtatg

SEQ ID NO: 12 - DNA, 19 bp, Artificial Sequence
  Reverse primer for ICAM
cctggcacattggagtctg

SEQ ID NO: 13 - DNA, 20 bp, Artificial Sequence
  Forward primer for VCAM
gaacccaaacaaaggcagag

SEQ ID NO: 14 - DNA, 19 bp, Artificial Sequence
  Reverse primer for VCAM
aggaagggctgaccaagac

SEQ ID NO: 15 - DNA, 20 bp, Artificial Sequence
  Forward primer for integrin beta 1 (ITGB1)
tgaatgggaacaacgaggtc

SEQ ID NO: 16 - DNA, 20 bp, Artificial Sequence
  Reverse primer for integrin beta 1 (ITGB1)
aattccagcaaccacaccag

(19) United States
(12) Patent Application Publication
(10) Pub. No.: US 2022/0259565 A1
(43) Pub. Date: Aug. 18, 2022

(54) Method of enhancing mobility of stem cells to inflammatory lesion

(71) Applicant: Gachon University of Industry-Academic Cooperation Foundation, Gyeonggi-do (KR)

(72) Inventors: Dongwoo KHANG, Seoul (KR); Jun-Young PARK, Incheon (KR); Jun Young PARK, Gyeonggi-do (KR)

(21) Appl. No.: 17/674,233

(22) Filed: Feb. 17, 2022

(30) Foreign Application Priority Data
Feb. 17, 2021 (KR) 10-2021-0020886

(51) Int. Cl. C12N 5/0775; A61P 43/00; A61K 35/28; A61P 29/02

(52) U.S. Cl. CPC C12N 5/0667; A61P 29/02; A61K 35/28; A61P 43/00

(57) ABSTRACT

The present invention elates to a novel method for enhancing the performance of stem cells, and more particularly, a method for enhancing the ability of stem cells to migrate to an inflammatory site, comprising culturing the stem cells in a culture medium of inflammation-related cells, and a therapeutic use of the stem cells having enhanced ability to migrate to an inflammatory site for treating inflammatory diseases or autoimmune diseases.

### CROSS-REFERENCE TO RELATED APPLICATION

This application claims priority to Korean Patent Application No. KR 10-2021-0020886 filed on Feb. 17, 2021 and all the benefits accruing therefrom under 35 U.S.C. § 119, the contents of which are incorporated by reference in their entirety.

### TECHNICAL FIELD

The present invention relates to a method of enhancing performance of stem cells, and more particularly, to a method of enhancing mobility of stem cells to an inflammatory lesion.

### BACKGROUND

Mesenchymal stem cells (hereinafter abbreviated as “MSCs”) are pluripotent cells that can differentiate into various mesenchymal tissues (Ullah et al., iScience, 15:421-438, 2019; Zuk et al., Mol. Biol. Cell, 13(12):4279-4295, 2002). Stem cells were first isolated from bone marrow and then isolated from fat, periodontal, muscle, dermis, and umbilical cord blood, and over the past decades MSCs have been studied in regenerative medicine and in various other technical fields (Zuk et al., Mol. Biol. Cell, 13(12): 4279-4295, 2002; Crisan et al., Cell Stem Cell, 3(3):301-313, 2008; Gronthos et al., Proc. Natl. Acad. Sci. U.S.A., 97(25): 13625-13630, 2000). The ability to move stem cells to the lesion site is very important to fully potentiate capabilities of stem cells in the regenerative medicine field, and the effect is maximized when MSCs injected into the blood vessel are targeted to the lesion through blood circulation (Kim et al., Adv. Sci. (Weinh), 5(5): 1700860, 2018). Exact mechanism of MSCs' targeting to the lesion has not yet been identified, but it is generally known that chemokines (CCL12) and their receptors (CXCR4), ICAM-cellin interactions, (VACM)-VLA4 interactions, and the extracellular matrix (hereinafter referred to as “ECM”) facilitates MSC delivery to various disease sites, by maximizing the target ability of these MSCs (Ullah et al., iScience, 15:421-438, 2019).

Steroid drugs are commonly used to treat rheumatoid arthritis, one of the autoimmune diseases, and repeated administration causes many side effects such as muscle weakness, osteoporosis, obesity, hypertension, and Cushing syndrome (Winblad et al., Rhinol. 55(3): 195-201, 2017; Eisenhofer et al., Clin. Chem., 64(3): 586-596, 2018). In addition, exacerbation of pain and inflammatory conditions caused by inflammatory cytokines released from macrophages and synovial fibroblasts in inflammatory immune diseases and arthritis were reported when steroid drugs are used (Xing et al., Scand. J. Imunol., 83(1):64-71, 2016; McInnes and Schett, Nat. Rev. Imunol., 7(6):42942, 2007).

For drug delivery systems using stem cells, their ability to migrate to the target site is important to confirm efficacy of drug being used in vivo, but due to the accumulation of a significant number of MSCs in the liver, spleen, and lung, stem cells engineered to improve the targeting ability of MSCs have been studied over the past few years (Bang et al., Cell Med., 4(2): 65-76, 2012; Cuiffo and Karnoub, Cell Adh. Migr., 6(3): 220-230, 2012).

The mobility of MSCs was improved by overexpression of chemokine receptors. It was reported that CXCR4 overexpression improved mobility in an animal model of cardiac infarction and improvement of the condition (Bang et al., Cell Med., 4(2): 65-76, 2012; Gao et al., Stem Cells, 27(4): 857-856, 2009; Lau and Wang, Expert Opin. Biol. Ther. 11(2): 189-197, 2011). In addition, the same effect was also demonstrated in an animal model of colitis. Specifically, it was confirmed that the return of damaged MSCs was promoted through overexpression of CXCR7 (Ren et al., J. Immunol., 184(5): 2321-2328, 2010; Xiao et al., Cell. Biochem. Biophys., 62(3): 409-414, 2012). There is also a report that overexpression of α-4 integrin, a component of VLA-4, also improved the mobility of MSCs (Bang et al., Cell Med., 4(2): 65-76, 2012).

### SUMMARY

However, in the case of genetically engineered stem cells, various side effects, such as inducing metastasis of breast cancer cells and differentiation into tumor stem cells due to mutation by genetic manipulation have been suggested (Cuiffo and Krnoub, Cell Adh. Migr 6(3): 220-230, 2012; Lin et al., Hindawi BioMed Res. Int., 2009, 2820853, 2019; Marofi et al., Front. Immunol., 8: 1770, 2017).

Therefore, the present invention has been devised to solve the various problems described above, and thus the object of the present invention is to provide a new method that can efficiently increase the ability of stem cells to migrate to inflammatory tissues without using unnecessary processes such as genetic manipulation. However, these objects are exemplary and do not limit the scope of the present invention.

According to one aspect of the present invention, there is provided a method for enhancing the ability of stem cells to migrate to an inflammatory site, comprising the step of culturing the stem cells in a culture medium of inflammation-related cells.

According to another aspect of the present invention, there is provided a pharmaceutical composition for treating an inflammatory disease or an autoimmune disease comprising stem cells having enhanced ability to migrate to an inflammatory site by the above method as an active ingredient.

According to another aspect of the present invention, there is provided a pharmaceutical composition for treating an inflammatory disease or autoimmune disease comprising a stem cell having enhanced mobility to an inflammatory site by the method and nanoparticles loaded with an anti-inflammatory agent bound to the stem cell as an active ingredient.

### BRIEF DESCRIPTION OF THE DRAWINGS

FIG. 1 is a schematic diagram illustrating an experimental method for confirming the mobility of various stem cells to inflammatory cells according to an embodiment of the present invention.

FIG. 2 is a graph quantifying and showing experimental results performed according to the experimental method illustrated in FIG. 1.

FIG. 3 is a schematic diagram schematically illustrating an experimental method for identifying a chemokine and a chemokine receptor affecting the mobility of AD-MSCs to inflammatory cells according to an embodiment of the present invention.

FIG. 4 is a series of fluorescent microscopic images showing results of measuring the mobility of AD-MSCs treated with various chemokine and chemokine receptor-specific antibodies (anti-CCR1, anti-CCR2, anti-CCR3, and anti-CXCR4) to TNF-α-stimulated FLS according to the experimental method illustrated in FIG. 3.

FIG. 5 is a series of fluorescent microscopic images showing the results of measuring the mobility of AD-MSCs treated with various chemokine and chemokine receptor-specific antibodies (anti-CCR1, anti-CCR2, anti-CCR3, and anti-CXCR4) to LPS-stimulated macrophages (J774) according to the experimental method illustrated in FIG. 3.

FIG. 6 is a graph quantifying and showing the results of experiments performed by according to experimental method illustrated in FIGS. 4 and 5.

FIG. 7 is a schematic diagram illustrating an experimental method for comparing the mobility of AD-MSCs pre-educated with TNF-α-stimulated fibroblast (FLS) to TNF-α-stimulated fibroblast FLS, with the mobility of non-educated AD-MSCs.

FIG. 8 is a schematic diagram schematically illustrating an experiment comparing the mobility of AD-MSCs pre-treated with LPS-stimulated macrophages (J774) which are inflammatory cells to LPS-stimulated J774.

FIG. 9 is a series of fluorescence microscopic images showing experimental results performed according to the experimental method illustrated in FIG. 7.

FIG. 10 is a graph quantifying and showing the result of FIG. 9.

FIG. 11 is a series of fluorescence microscope photographs showing experimental results performed according to the experimental method of FIG. 8.

FIG. 12 is a graph quantifying and showing the result the result of experiment performed by according to experimental method illustrated in FIG. 11.

FIG. 13 is a schematic diagram schematically illustrating an experimental method for determining whether a stem cell education method according to an embodiment of the present invention is specific to inflammatory cells used in education.

FIG. 14 is a series of fluorescence microscope photographs showing experimental results performed according to the experimental method illustrated in FIG. 13.

FIG. 15 is a graph quantifying and showing the result of experiment performed by according to experimental method illustrated in FIG. 14.

FIG. 16 is a graph showing the results of quantifying the expression levels of various cell markers, chemokines, and chemokine receptors in AD-MSCs pre-educated with inflammatory cells compared with non-educated AD-MSCs according to an embodiment of the present invention.

FIG. 17 shows a hitmap (left panel) regarding genes whose expressions are increased at the mRNA level and the ranking and list of the top 100 genes with increased expression (right panel) in AD-MSCs pre-educated with inflammatory cells compared with non-educated control AD-MSCs according to an embodiment of the present invention.

FIG. 18 is an overview of an experiment for confirming the performance of a target-enhanced stem cell according to an embodiment of the present invention using an arthritis model and a series of photographs showing whether the stem cell moves to an arthritis area after administering the targeting-enhanced stem cells to an experimental animal according to an embodiment to the present invention.

### DETAILED DESCRIPTION OF THE INVENTION

### Definition of Terms

The term “inflammatory-related cells” used herein are cells present in the body which initiate or mediate inflammatory reaction, collectively referring to immune cells or non-immune cells present in the inflammatory site. Among these inflammatory-related cells, immune cells include macrophages, neutrophils, eosinophils, and lymphocytes, and non-immune cells include keratinocytes and synovial fibroblasts.

The term “inflammation-inducing substance” used herein means any substance that initiates or amplifies and spreads inflammatory reactions, such as pro-inflammatory cytokines secreted by immune cells in the body, debris of apoptotic or necrotic cells, and substances released from infected pathogens. Inflammatory cytokines include TNF-α, IL-1β, IL-6, IL-8, IL-12, GM-CSF, INF-γ, and IL-18, and substances released from the infected pathogens, i.e., pathogen-derived inflammation-inducing substances, include typically lipopolysaccharides (LPS) which are cell wall components of Gram-negative bacteria and act as endotoxins.

The term “nanoparticle” used herein refers to a particle having a size of several to hundreds of nanometers (nm). The nanoparticles may be formed of various materials such as metal, phospholipid, biodegradable polymer, carbon nanotube, fullerene, carbon nanodot, or the like, or a composite of any two or more of them. The nanoparticles may have a surface coated with a biocompatible material, or may have a stem cell surface marker-specific antibody attached for binding to stem cells. In addition, in various ways, a drug-complex may be formed in a form in which the drug is enclosed inside or attached to the surface by a covalent/non-covalent bond.

### DETAILED DESCRIPTION OF THE INVENTION

According to one aspect of the present invention, there is provided a method for enhancing the ability of stem cells to migrate to an inflammatory site, comprising the step of culturing the stem cells in a culture medium of inflammation-related cells.

In the method, the inflammation-related cells may be synovial fibroblasts, inflammation-related macrophages, neutrophils, eosinophils, lymphocytes, or keratinocytes.

In the above method, the inflammation-related cells may be isolated from the inflammatory site of the patient, or may be the same type of cells stimulated with an inflammation-inducing substance as those of isolated from the inflammatory site of the patient. Optionally, the inflammation-related cells separated from the inflammatory site of the patient may also be stimulated with an inflammation-inducing substance. The inflammation-inducing substance may be pro-inflammatory cytokine, inflammatory chemokine, or a pathogen-derived inflammatory substance, and the inflammatory cytokine may be TNF-α, IL-1β, IL-6, IL-8, IL-12, GM-CSF, INF-γ, and IL-18 and the inflammatory chemokine may be MCP-4. The inflammation-inducing substance may be endotoxin or exotoxin, all of which may be a toll-like receptor (TLR) ligand. TLR ligands include lipopolysaccharides (hereinafter abbreviated as “LPS”) as endotoxins, which are representative inflammation-inducing substances known as TLR4 ligands, triacylated lipoprotein, a TLR1 or TLR2 ligand; zymosan, a TLR6 ligand; flagellin, a TLR6 ligand; dsRNA, a TLR3 ligand; ssRNA, a TLR7 or TLR8 ligand; and CpG oligodeoxyriborucleotide (ODN), TLR9 ligand. The inflammation may be an infectious inflammation caused by a pathogen such as bacteria or viruses, or may be one caused by an autoimmune disease caused by the collapse of the Th1/Th2 balance, such as rheumatoid arthritis or atopic dermatitis. In particular, in the former case, stimulation methods using bacterial inflammatory substances such as LPS may be suitable for pathogenic inflammation model, and in the latter case, stimulation methods using inflammatory cytokines such as TNF-α may be more suitable for autoimmune diseases.

In the above method, the stem cells may be adipose-derived stem cells, umbilical cord blood-derived stem cells, bone marrow-derived stem cells, dental pulp-derived stem cells, muscle-derived stem cells, or dermal stem cells, preferably adipose-derived stem cells, but are not limited thereto.

According to another aspect of the present invention, there is provided a pharmaceutical composition for treating an inflammatory disease or an autoimmune disease comprising stem cells having enhanced ability to migrate to an inflammatory site prepared by the above-described method as an active ingredient.

In the pharmaceutical composition, the stem cells having enhanced ability to migrate to an inflammatory site may be stem cells having at least four times increased mobility to an inflammatory cells as compared with an uneducated stem cells, or a stem cell having at least 1.5 times or at least twice increased mobility to inflammatory cells as compared with stem cells not treated with an inflammation-inducing substance.

In the pharmaceutical composition, the stem cells having enhanced ability to migrate to an inflammatory site may be stem cells whose expression of ICAM and VCAM is increased 20-fold or more at the mRNA level compared to un-educated stem cells.

In the pharmaceutical composition, the stem cells having enhanced ability to migrate to an inflammatory site may be stem cells educated with culture medium of synovial fibroblasts treated with TNF-α, and whose expression level of ICAM is at least 80 times higher, expression level of VCAM is increased at least 100 times, and expression level of CXCR4 may be at least 10 times higher than those of un-educated stem cells at the mRNA level.

In the pharmaceutical composition, the stem cells having enhanced ability to migrate to an inflammatory site may be stem cells educated with culture medium of inflammation-related macrophages or M1 macrophages treated with LPS, and whose expression level of ICAM is increase 20-fold or more compared with un-educated stem cells at the mRNA level.

In the pharmaceutical composition, the stem cells having enhanced ability to migrate to an inflammatory site may be stem cells whose expression level of at least one gene, or at least 20 to 50 genes, at least 40 to 70 genes, at least 50 to 80 genes, or at least 60 to 60 genes selected from the group of consisting of CXCL8, IL1B, CXCL5, CXCL3, CCL8, CXCL2, MMP1, IL6, CXCL1, CXCL10, CCL5, CXCL6 on an mRNA basis, CCL11, DNER, CCL7, CCL2, CXCL11, CXCL11, ICAM1, CCL3, VCAM1, NR4A2, TNFAIP3, CCL20, MMP12, LBP, SPATA13, EDNRB, PDE4D, CCL4L2, S PREX1, CYP7B1, PLCN4V, EFNA1, EFNA1, RHOU, EDN1, NR4A1, FGF1 3, SEMA4D, APCDD1, LAMBS, LTB4R2, ITGB3, ADGRG1, JUP, FYN, SLC7A7, ITGB8, CCL19, PDE4B, BDKRB4, WNT5 ADDIT, PDE4B, BDKITRB4, TGFB2, A GRB14, NDNF, RND3, PF4, GYPC, DPP4, FMNL1, PSTPIP2, ITGA1, NRP2, LCP1, CXCL9, JAM2, MSX2, PECAM1, HBEGF, LURAP1, PLXNB3, ANGPT1, ANGPT1, SLAMF8, SLC7A11, TNSLC3, SLC7A11, SDC4, ACKR3, PTN, LYST, EPHA4, STAT1, S1PR1, SEMA6C, SLC3A2, BAMBI, WWC1, OLR1, ZEB2, PARP9, SEMA3F, SEMA3F, CD34, BTG1, and SEMA4B is increase at least 5-fold compared with uneducated stem cells, or, optionally stem cells whose expression level of at least 2, 3, 4, 5, 6, 7, 8, 9, 10, 11, 12, 13, 14, 15, 16, 17, 18, 19, 20, 21, 22, 23, 24, 25, 26, 27, 28, 29 Dogs, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100 genes among the afore-mentioned genes may be increased by at least 5-fold compared with uneducated stem cells.

In the pharmaceutical composition, wherein the stem cells having enhanced ability to migrate to an inflammatory site may be stem cells educated with culture medium of synovial fibroblasts treated with TNF-α or inflammation-related macrophages or M1 macrophages treated with LPS.

The pharmaceutical composition may further include at least one or more anti-inflammatory agents.

In the pharmaceutical composition may further comprise at least one anti-inflammatory agent.

In the pharmaceutical composition, the anti-inflammatory agent may be provided simply in combination with the stem cells, or is optionally formulated separately and administered simultaneously or separately, or may be optionally loaded with a nanoparticle and when loaded on a nanoparticle, the nanoparticle may be may be attached to the surface of the stem cells or loaded inside the stem cells.

In the pharmaceutical composition, the stem cells may be adipose-derived stem cells, umbilical cord blood-derived stem cells, bone marrow-derived stem cells, dental pulp-derived stem cells, muscle-derived stem cells, dermal stem cells or it may be induced pluripotent stem cells (iPSCs).

In the pharmaceutical composition, the inflammatory disease may be rhinitis, allergic conjunctivitis, epidemic conjunctivitis, hepatitis, bronchitis, laryngitis, tonsillitis, thyroiditis, laryngitis, encephalitis, myelitis, pneumonia, gastritis, colitis, cystitis, pancreatitis, cystitis, synovitis, rheumatoid arthritis, osteoarthritis, ankylosing spondylitis, psoriasis, pruritus, pruritus, seborrheic dermatitis, acne, irritant dermatitis or atopic dermatitis. In addition, the autoimmune disease may be inflammatory bowel disease, rheumatoid arthritis, osteoarthritis, inflammatory myopathy, autoimmune vasculitis, autoimmune hepatitis, autoimmune pancreatitis, autoimmune encephalitis, autoimmune vasculitis, Behcet's disease, systemic lupus, Sjögren's syndrome, myasthenia gravis, scleroderma, polyarteritis nodosa, Kikuchi disease, collagen disease, Hashimoto's thyroiditis, vitiligo, Still's disease, alopecia areata, multiple sclerosis, orthostatic tachycardia syndrome, It may be autoimmune hemolytic anemia, Stevens-Jones syndrome, Galen-Barré syndrome, cytokine storm or pemphigus, and the inflammatory bowel disease may be ulcerative colitis or Crohn's disease.

In the pharmaceutical composition, the stem cells having enhanced ability to migrate to an inflammatory site may be stem cells educated with culture medium of synovial fibroblasts treated with TNF-α or inflammation-related macrophages or M1 macrophages treated with LPS.

The pharmaceutical composition may further include at least one or more anti-inflammatory agents.

In the pharmaceutical composition, the anti-inflammatory agent may be provided simply in combination with the stem cells, or may be optionally formulated separately and administered simultaneously or separately, or may be optionally loaded with nanoparticles. When the anti-inflammatory agent is loaded on the nanoparticles, the nanoparticles may be attached to the surface of the stem cells or loaded inside the stem cells.

In the pharmaceutical composition, the stem cells may be adipose-derived stem cells, umbilical cord blood-derived stem cells, bone marrow-derived stem cells, pulp-derived stem cells, muscle-derived stem cells, dermal-derived stem cells or induced pluripotent stem cells (iPSC).

In the pharmaceutical composition, the inflammatory disease may be rhinitis, allergic conjunctivitis, epidemic conjunctivitis, hepatitis, bronchitis, laryngitis, tonsillitis, thyroiditis, laryngitis, encephalitis, myelitis, pneumonia, gastritis, colitis, cystitis, pancreatitis, cystitis, synovitis, rheumatoid arthritis, osteoarthritis, ankylosing spondylitis, psoriasis, pruritus, pruritus, seborrheic dermatitis, acne, irritant dermatitis or atopic dermatitis. In the pharmaceutical composition, the autoimmune disease may be inflammatory bowel disease, rheumatoid arthritis, osteoarthritis, inflammatory myopathy, autoimmune vasculitis, autoimmune hepatitis, autoimmune pancreatitis, autoimmune encephalitis, autoimmune vasculitis, Behcet's disease, systemic lupus, Sjögren's syndrome, myasthenia gravis, scleroderma, polyarteritis nodosa, Kikuchi disease, collagen disease, Hashimoto's thyroiditis, vitiligo, Still's disease, alopecia areata, multiple sclerosis, orthostatic tachycardia syndrome, autoimmune hemolytic anemia, Stevens-Jones syndrome, Galen-Barré syndrome, cytokine storm or pemphigus, and the inflammatory bowel disease may be ulcerative colitis or Crohn's disease.

In the pharmaceutical composition, the anti-inflammatory agent may be a corticoid-based anti-inflammatory agent or a non-steroid anti-inflammatory agent (NSAID), and the corticoid-based anti-inflammatory agent may be hydrocortisone, hydrocortisone acetate, cortisone, cortisone acetate, tixocortol pivalate, hydrocortisone-17-valerate, halometasone, alclometasone dipropionate, betamethasone valerate, betamethasone dipropionate, prednicarbate, clobetasone-17-butyrate, clobetasol-17-propionate, fluocortolone caproate, fluocortolone pivalate, fluprednidene acetate, prednisone, prednisolone, methylprednisolone, dexamethasone, dexamethasone sodium phosphate, betamethasone, betamethasone sodium phosphate, fluocortolone, triamcinolone, triamcinolone acetonide, mometasone, amcinonide, desonide, fluocinonide, fluocinolone acetonide, halcinonide, beclomethasone, fludrocortisone acetate, hydrocortisone-17-butyrate, hydrocortisone aceponate, hydrocortisone ybuteprate, ciclesonide, or prednicarbate. In the pharmaceutical composition, the non-steroid anti-inflammatory agent may be a cyclooxygenase (COX) inhibitor, and the cyclooxygenase inhibitor may be a non-selective COX-1/COX-2 inhibitor, a selective COX-1 inhibitor or a selective COX-2 inhibitor. The selective COX-2 inhibitor may be apricoxib, celecoxib, rofecoxib, parecoxib, lumiracoxib, etoricoxib, or pyrocoxib.

In addition, in the pharmaceutical composition, the nanoparticle may be gold nanoparticle, carbon nanotube, liposome, exosome, or nanoparticle having a core/shell structure including a biodegradable polymer, and the nanoparticle may be attached on the surface of the stem cells via an antibody specific for a stem cell-specific surface marker, which is attached to the surface of the nanoparticle.

The composition may include a pharmaceutically acceptable carrier, and may additionally include a pharmaceutically acceptable adjuvant, excipient or diluent in addition to the carrier.

As used herein, the term “pharmaceutically acceptable” refers to a composition that is physiologically acceptable and does not normally cause gastrointestinal disorders, allergic reactions such as dizziness, or similar reactions when administered to humans. Such carriers, excipients and diluents may include lactose, dextrose, sucrose, sorbitol, mannitol, xylitol, erythritol, maltitol, starch, gum acacia, alginate, gelatin, calcium phosphate, calcium silicate, cellulose, methyl cellulose, polyvinylpyrrolidone, water, methylhydroxybenzoate, propylhydroxybenzoate, talc, magnesium stearate, and mineral oil. In addition, fillers, anti-agglomeration agents, lubricants, wetting agents, flavoring agents, emulsifiers and preservatives and the like may be included in the pharmaceutical composition.

In addition, the pharmaceutical composition according to an embodiment of the present invention may be formulated using a method known in the art to enable rapid, sustained or delayed release of the active ingredient when administered to a mammal. The formulations may include powders, granules, tablets, emulsions, syrups, aerosols, soft or hard gelatin capsules, sterile injectable solutions, and sterile powder forms.

The composition according to an embodiment of the present invention may be administered by various routes, for example, oral, parenteral, for example, suppository, transdermal, intravenous, intraperitoneal, intramuscular, intralesional, intranasal, intrathecal administration, and may also be administered using an implantable device for sustained release or continuous or repeated release. The number of administration may be once a day or divided into several times within a desired range, and the pharmaceutical composition may be administered at intervals such as once a week, twice a week, once a month, and the administration period is not particularly limited.

The composition according to an embodiment of the present invention may be formulated in a suitable form together with a commonly used pharmaceutically acceptable carrier. Pharmaceutically acceptable carriers include, for example, carriers for parenteral administration such as water, suitable oils, saline, aqueous glucose and glycol, and the like, and may further include stabilizers and preservatives. Suitable stabilizers include antioxidants such as sodium hydrogen sulfite, sodium sulfite or ascorbic acid. Suitable preservatives may be benzalkonium chloride, methyl- or propyl-paraben and chlorobutanol. In addition, the composition according to the present invention may further comprise a suspending agent, solubilizing agent, stabilizer, isotonic agent, preservative, adsorption inhibitor, surfactant, diluent, excipient, pH adjuster, analgesic agent, buffer, and antioxidants if necessary. Pharmaceutically acceptable carriers and agents suitable for the present invention, including those exemplified above, are described in detail in Remington's Pharmaceutical Sciences, latest edition.

The dosage of the composition to a patient will depend on many factors including the patient's height, body surface area, age, the particular compound being administered, sex, time and route of administration, general health, and other drugs being administered concurrently. The pharmaceutically active protein may be administered in an amount of 100 ng/body weight (kg)-10 mg/body weight (kg), more preferably 1 to 500 μg/kg (body weight), and most preferably, it may be administered in an amount of 5 to 50 μg/kg (body weight), and the dosage may be adjusted in consideration of the above factors.

According to another aspect of the present invention, there is provided a pharmaceutical composition for treating an inflammatory disease or autoimmune disease comprising a stem cell having enhanced mobility to an inflammatory site by the method and nanoparticles loaded with an anti-inflammatory agent bound to the stem cell as an active ingredient.

In the method, the inflammatory disease and autoimmune disease are as described above.

As used herein, the term “therapeutically effective amount” means an amount sufficient to treat a disease with a reasonable benefit/risk ratio applicable to medical treatment, and the effective dose level depends on the type and severity of the subject, age, sex, drug activity, sensitivity to drug, administration time, administration route and excretion rate, duration of treatment, factors including co-administrated drugs and other factors well known in the medical field. A therapeutically effective amount of the composition of the present invention may be 0.1 mg/kg to 1 g/kg, more preferably 1 mg/kg to 500 mg/kg, but the effective dosage may be appropriately adjusted depending on the age, sex and condition of the patient.

Hereinafter, the present invention will be described in more detail through following examples. However, the present invention is not limited to the examples described below, and the examples are provided to disclose fully disclose the present invention and to inform those skilled in the art the scope of the invention.

### EXAMPLE

Common Methods

Cell Lines and Cell Cultures

Fibroblast-like synoviocytes (FLSs) were isolated from rheumatoid patients at Kyungpook National University in Korea, and only cells within 5 passages (P5) were used. The FLSs were cultured in Dulbecco's Modified Eagle Medium (DMEM, Cat No.: 11965084) supplemented with 10% fetal bovine serum (Cat No.: 16000044) and 1% penicillin (Cat No.: 15140163), and the above reagents were all purchased from Gibco (USA)

J774 A.1 cells were purchased from the American Type Culture Collection (ATCC). It was cultured in DMEM supplemented with 10% fetal bovine serum and 1% penicillin and all the reagents were purchased also from Gibco (USA).

Stem cells used herein were bone marrow-derived stem cells (BM-MSCs), adipose-derived stem cells (AD-MSCs), and umbilical cord blood-derived stem cells (UC-MSCs) and they were purchased from CEFO Co., Ltd. (Korea). Cells were cultured using a bio-stem cell growth culture medium, and only cells within 5 passages were used for all stem cells.

Vascular endothelial cells (HUVECs) were purchased from CEFO Co., Ltd. (Korea) and cultured using HUVEC growth culture medium (CEFO Co., Ltd., Korea), and all experiments were performed in the logarithmic growth stage.

### Example 1: Ability of Various Stem Cells to Migrate into Target Inflammatory Cells

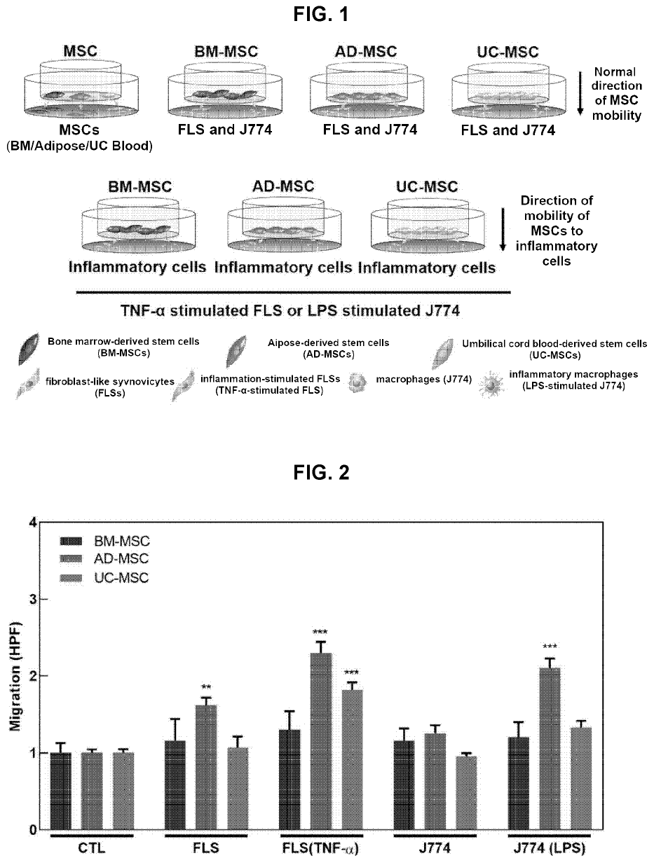

In cancer cells, various initial inflammatory responses are induced and various immune cells migrate. These immune cells in turn express various chemokines. Accordingly, the present inventors tried to confirm the migration of stem cells using chemokines discovered through prior research in an inflammation model. As a result, the present inventors tried to confirm the migration ability of stem cells using macrophages, which typically induce chronic inflammation, and synovial fibroblasts, which induce arthritis inflammation, as model cells. The present inventors intended to confirm whether the phenotype of inflammatory synovial fibroblasts appears when TNF-α was treated to synovial fibroblast isolated from rheumatoid arthritis site of rheumatoid arthritis patients, and whether undifferentiated macrophages can be differentiate into M1 inflammatory macrophages when treated with LPS (FIG. 1).

In order to confirm the above, the present inventors specifically dispensed 5×10⁴ cells of FLSs and J774A.1 cells in a volume of 500 μl in a Corning® 24-well plate, respectively. The next day, FLSs were treated with TNF-α (20 ng/ml) and J774A.1 cells were treated with LPS (50 ng/ml) to induce an inflammatory state. The next day, 5×10⁴ stem cells (BM-MSCs, AD-MSCs, and UC-MSCs) each were dispensed in a volume of 200 μl into Corning® TransWell® Polycarbonate membrane cell culture inserts, and the cell culture insets were inserted to plates in which the cells cultured in an inflammatory state. After inserting the insert, additional culture was performed for 3 hours, and then fixed with methanol at −20° C. for 1 minute. Then, after washing with PBS 3 times, DAPI staining was performed, and cell migration was confirmed by imaging using EVOS M7000 imaging system.

As a result, as shown in FIG. 2, it was confirmed that the ability of stem cells to migrate to FLSs or J774 cells in an inflammatory state was significantly increased in the case of adipose-derived stem cells among various stem cells. In the case of bone marrow-derived stem cells, no improvement in mobility was observed in both non-stimulated synovial fibroblasts and macrophages, as well as inflammation-stimulated synovial fibroblasts and macrophages. On the other hand, in the case of umbilical cord-derived stem cells, the improvement of ability to migrate into FLSs was observed, whereas a significant result could not be obtained in macrophages.

### Example 2: Experiments to Identify Markers for Each Inflammatory Cell Using Anti-Chemokine Receptors

From the results of Example 1, the present inventors tried to confirm whether there is a difference in cell markers depending on the cell type of the inflammation-inducing cells using an antibody capable of blocking the action of various chemokines and chemokine receptors.

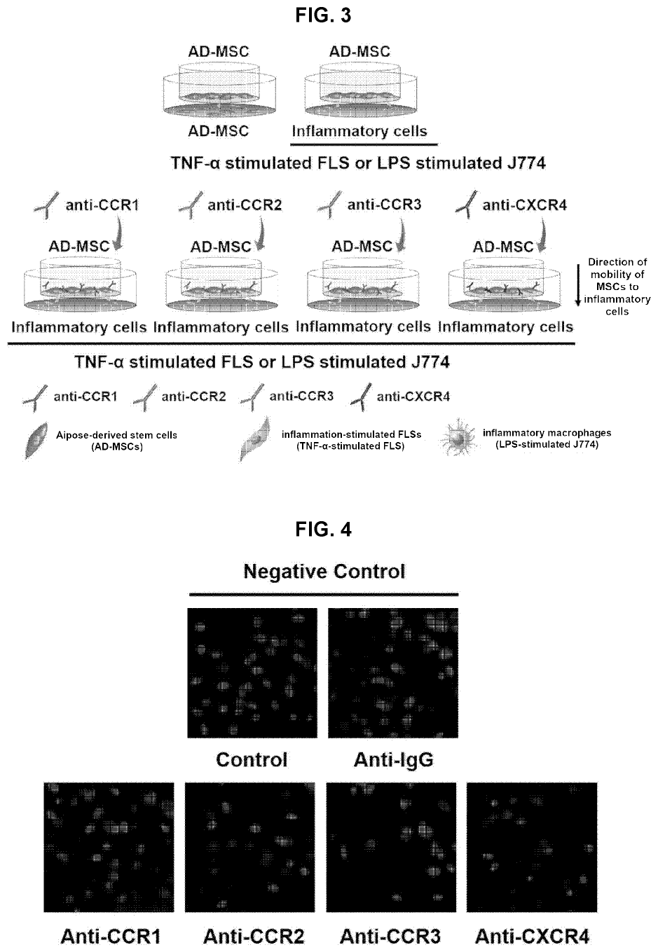

Specifically, the present inventors dispensed 5×10⁴ cells each of FLSs, J774A.1 cells, and AD-MSCs in a volume of 500 μl in a Corning® 24-well plate. The next day, FLSs were treated with TNF-α (20 ng/ml), and J774A.1 cells were treated with LPS (50 ng/ml). The next day, stem cells were treated with anti-CCR1 antibody, anti-CCR2 antibody, anti-CCR3 antibody, and anti-CXCR4 antibody, respectively. The cells were dispensed in a volume of 200 μl into Corning® TransWell® Polycarbonate membrane cell culture inserts, and the cell culture inserts were inserted into the plate in which the inflammatory cells were cultured and further cultured for 3 hours. All antibodies used above were purchased from abcam (ab89055, ab203128, ab16231, and ab124824, respectively). After completing the additional culture, it was fixed with methanol at −20° C. for 1 minute. After washing with PBS 3 times, DAPI staining was performed, and the stem cell migration ability was measured by imaging using EVOS M7000 imaging system (FIG. 3).

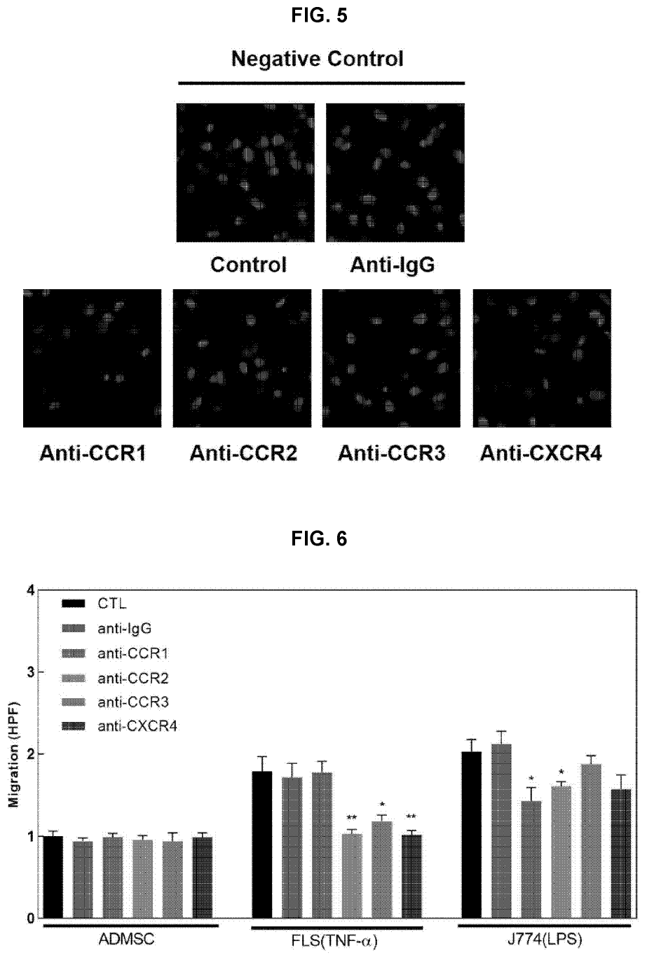

As a result, it was confirmed that there is a difference in cytokines and chemokines expressed depending on the type of inflammatory cells, specifically, there is a difference in the chemokine receptors that target inflammatory cells. Specifically, the present inventors confirmed that CCR2, CCR3, and CXCR4 act as migratory markers for inflammation-inducing synovial fibroblasts (FIG. 4). It was confirmed that CCR1, CCR2, CCR3, and CXCR4 were involved in migration to inflammatory M1 macrophages. (FIG. 5). The reduction in the mobility of these stem cells was statistically analyzed (FIG. 6).

### Example 3: Stem Cell Targeting Ability Improvement Experiment Using Non-Genetic Manipulation Education

From the results of Example 2, the present inventors tried to confirm whether or not the ability to migrate to inflammation-inducing cells is enhanced when pre-educating stem cells by co-culturing with inflammation-inducing cells.

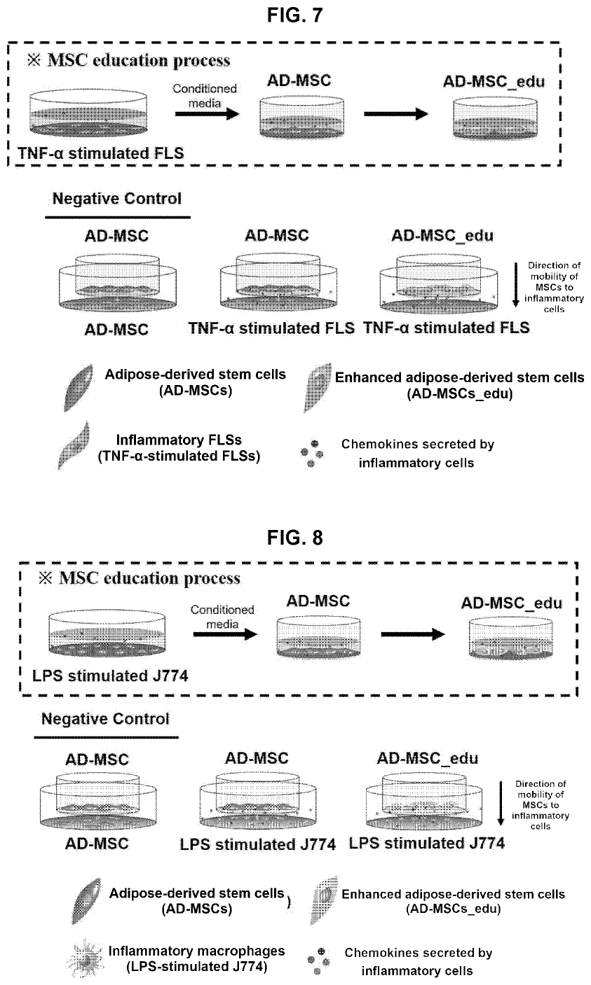

Specifically, the present inventors dispensed 2×10⁵ FLSs, J774A.1 cells, and AD-MSCs into Corning® 6-well plates, respectively. The next day, FLSs were treated with TNF-α (20 ng/ml), and J774A.1 cells were treated with LPS (50 ng/ml). The next day, the culture medium of FLSs, J774A.1 cells were treated to AD-MSCs, respectively, and the AD-MSCs were educated for 24 hours using the culture medium of the inflammatory cells. The next day, 5×10⁴ cells of inflammatory cells were each aliquoted in a volume of 500 μl in a Corning® 24-well plate. The next day, 5×10⁴ stem cells and 5×10⁴ each of the educated stem cells were aliquoted into Corning® TransWell® Polycarbonate membrane cell culture inserts in a volume of 200 μl, and the stem cells were inserted into the culture plate in which the inflammatory cell were cultured and further cultured for 3 hours. After the additional culture was completed, it was fixed with methanol at −20° C. for 1 minute. Then, after washing 3 times with PBS, DAPI staining was performed, and stem cell migration ability was measured by imaging using EVOS M7000 imaging system (FIGS. 7 and 8).

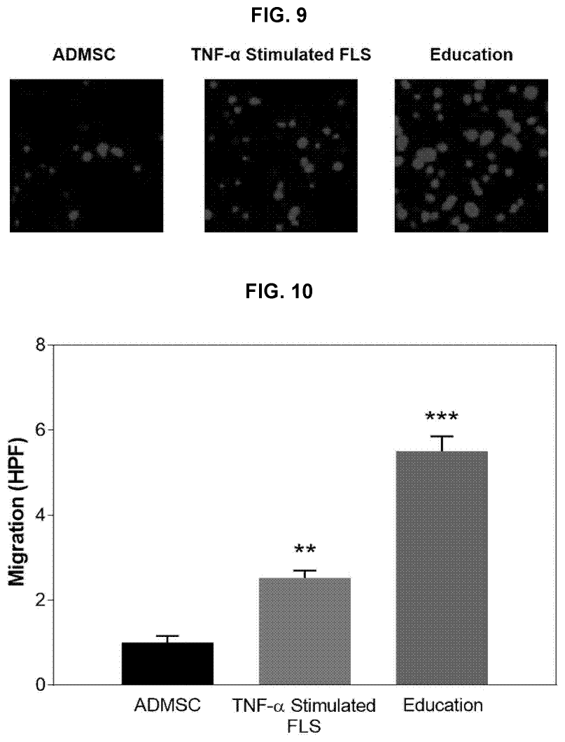

As a result, it was confirmed that stem cells (MSCs) pre-educated with synovial fibroblasts had improved migration ability compared to un-educated stem cells (FIG. 9). After statistical processing, the ability of pre-educated AD-MSCs according to an embodiment of the present invention to migrate to TNF-α-stimulated synovial fibroblasts was increased 5.5-fold compared to the migration ability of un-educated stem cells to AD-MSCs not treated with TNF-α and at least 2-fold compared to the migration ability of un-educated AD-MSCs to TNF-α-stimulated synovial fibroblasts (FIG. 10).

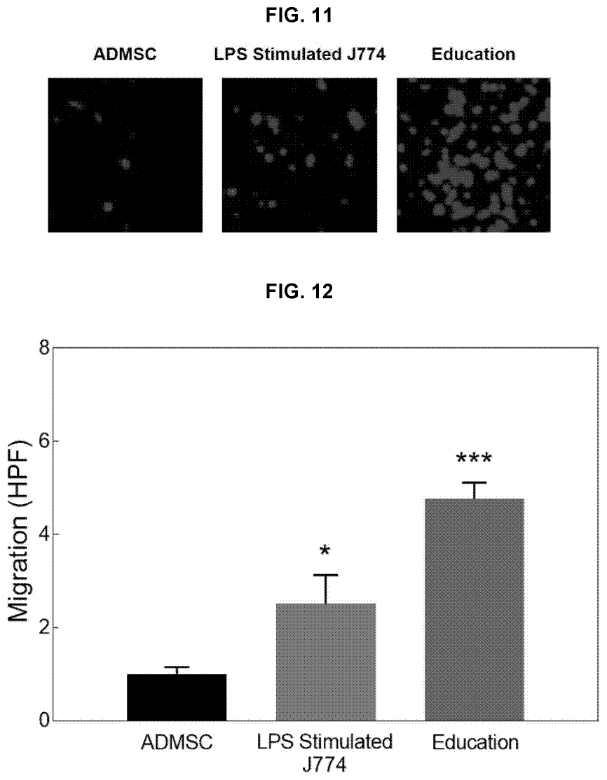

In addition, pre-educated AD-MSCs co-cultured with macrophages also had improved migration ability compared to un-educated AD-MSCs (FIG. 11). After statistical processing, the ability of pre-educated AD-MSCs according to an embodiment of the present invention to migrate to LPS-stimulated macrophages was increased 4.7-fold compared to the migration ability of un-educated stem cells to AD-MSCs not treated with LPS and at least 1.8-fold compared to the migration ability of un-educated AD-MSCs to LPS-stimulated macrophages (FIG. 12).

### Example 4: Investigation of the Ability of Stem Cells to Migrate to Another Types of Educated Inflammatory Cells

Based on the results of Example 3, in order to confirm whether the enhancement of the migration ability of pre-educated stem cells to inflammatory cells is a phenomenon specific to the inflammatory cells used in the education or a phenomenon that acts equally on pan-inflammatory cells. The experiment was performed in the same manner as in Example 3 by changing the inflammatory cells used for education and the inflammatory cells used for the evaluation of actual mobility.

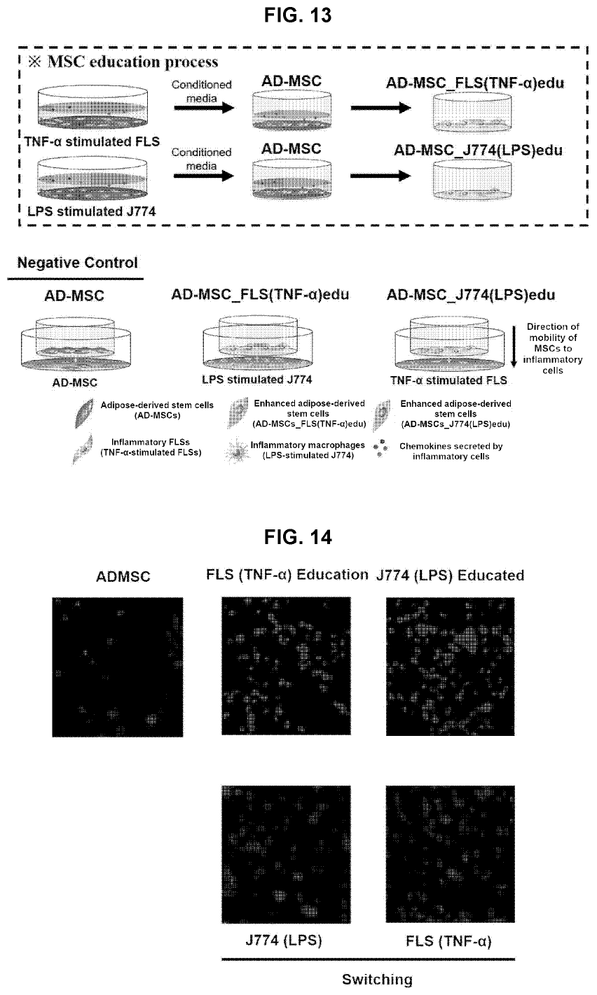

Specifically, the present inventors dispensed 2×10⁵ FLSs, J774 cells, and AD-MSCs into Corning® 6-well plates, respectively. The next day, FLSs were treated with TNF-α (20 ng/ml), and J774A.1 cells were treated with LPS (50 ng/ml). The next day, cell culture media of FLSs and J774A.1 were treated with AD-MSCs, and the AD-MSCs were educated for 24 hours using the culture medium of the inflammatory cells. The next day, 5×10⁴ cells of inflammatory cells were each aliquoted in a volume of 500 μl in a Corning® 24-well plate. The next day, 5×10⁴ stem cells and 5×10⁴ each of the educated stem cells were aliquoted into Corning® TransWell® Polycarbonate membrane cell culture inserts in a volume of 200 μl. At this time, unlike Example 3, the migration ability of inflammatory cells other than those used for pre-education was evaluated (FIG. 13). After 3 hours, the AD-MSCs were fixed with methanol at −20° C. for 1 minute. Thereafter, after washing with PBS 3 times, DAPI staining was performed, and migration of stem cells was measured by imaging using EVOS M7000 imaging system.

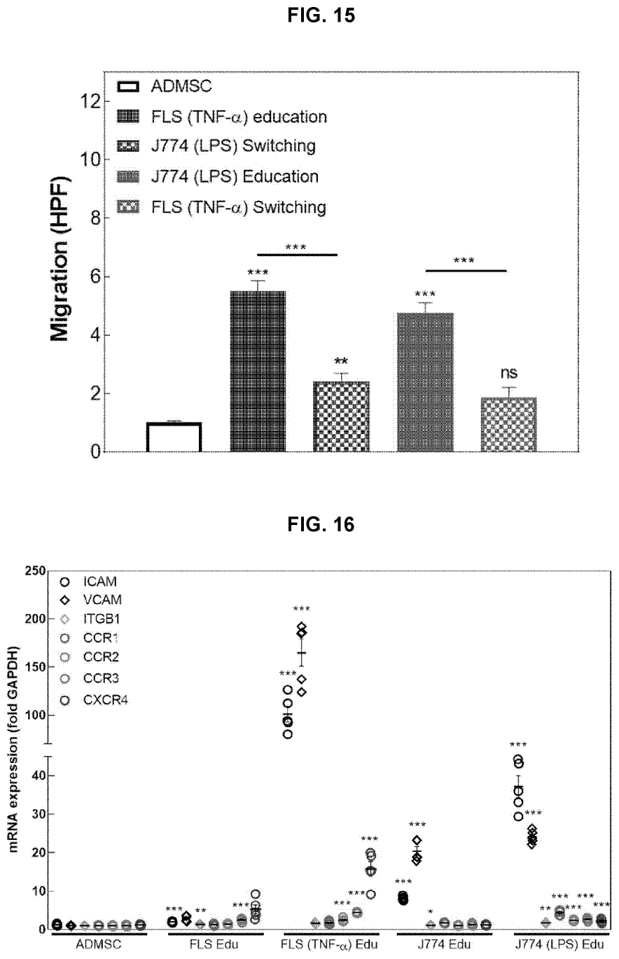

As a result, adipose-derived stem cells pre-educated with inflammatory macrophages (J774A.1) increased their migration ability into inflammatory-stimulated synovial fibroblasts (FLSs), but showed a lesser extent than adipose-derived stem cells pre-educated with inflammatory-stimulated synovial fibroblasts and the migration ability of adipose-derived stem cells pre-educated with inflammatory-stimulated synovial fibroblasts (FLS) into inflammatory macrophages (J774A.1) did not increase (FIGS. 14 and 15). Therefore, it was found that the improvement of the migration ability of stem cells into inflammatory cells was specific to the type of inflammatory cells used in education.

### Example 5: Identification of Stem Cell-Inflammatory Cell Chemokine Receptors and Mobility Markers Using Quantitative Real-Time PCR

In order to investigate the mechanism of improvement in cell migration ability after education, which is a non-genetic manipulation, gene expression levels of representative genes related to cell migration (ICAM, VCAM, and integrin beta 1 (ITGB1)) and chemokine receptors (CCR1, CCR2, CCR3, and CXCR4) were analyzed by quantitative real-time PCR.

To this end, the present inventors specifically seeded 2×10⁵ cells of FLSs, J774A1 cells, and AD-MSCs in Corning® 6-well plates, respectively. The next day, by treating culture media of the FLSs, J774A.1 cells to AD-MSCs, pre-education was carried out for 24 hours. Then, RNA was extracted from AD-MSCs. RNA extraction was performed with TRIzol reagent (Cat. No.: 15596018) purchased from Thermo Fisher Scientific. Thereafter, cDNA was synthesized using 1 μg/ml RNA. Reverse transcriptase (M1705) used for cDNA synthesis was purchased from Promega (USA). Quantitative real-time PCR results were measured with Bio-Rad CFX 384. SYBR® Green Maser (ROX, Cat. No.: 04913914001) was purchased from Roche (Swiss). All experiments were performed according to the manufacturers' instructions. The nucleic acid sequences of primers used in quantitative real-time PCR are shown in Table 1 below.

TABLE 1
Primer information used for quantitative RT-PCR

| Target Genes | Nucleotide sequences | SEQ ID NOs. |
|---|---|---|
| GAPDH | Foward: 5′-GTA TGA CAA CGA ATT TGG CTA CAG-3′ | 1 |
| | Reverse: 5′-TCT CTC TCT TCC TCT TGT GCT CTT-3′ | 2 |
| CCR1 | Foward: 5′-GAA ACA TCC TGG TGG TCC TG-3′ | 3 |
| | Reverse: 5′-AAG-AGC-AGG-TCA-GAA-ATG-GC-3′ | 4 |
| CCR2 | Foward: 5′-AGC TGA AGT GCT TGA CTG AC-3′ | 5 |
| | Reverse: 5′-TTG CAT TCC CAA AGA CCC AC-3′ | 6 |
| CCR3 | Foward: 5′-GGG CAG ATA CAT CCC ATT CC-3′ | 7 |
| | Reverse: 5′-ACA CAA TAG AGA GTT CCG GC-3′ | 8 |
| CXCR4 | Foward: 5′-AAA TCT TCC TGC CCA CCA TC-3′ | 9 |
| | Reverse: 5′-ACT TGT CCG TCA TGC TTC TC-3′ | 10 |
| ICAM | Foward: 5′-GGA GCT TCG TGT CCT GTA TG-3′ | 11 |
| | Reverse: 5′-CCT GGC ACA TTG GAG TCT G-3′ | 12 |
| VCAM | Foward: 5′-GAA CCC AAA CAA AGG CAG AG-3′ | 13 |
| | Reverse: 5′-AGG AAG GGC TGA CCA AGA C-3′ | 14 |
| integrin beta 1 | Foward: 5′-TGA ATG GGA ACA ACG AGG TC-3′ | 15 |
| (ITGB1) | Reverse: 5′-AAT TCC AGC AAC CAC ACC AG-3′ | 16 |

As a result, it was confirmed that the expression of the chemokine receptor was increased when the pre-education was performed with the inflammatory cells rather than the pre-education with the synovial fibroblasts and macrophages which were not stimulated with inflammation condition (FIG. 16).

This suggests that migration ability of stem cells to the inflammatory cells can be improved because the expression of the chemokine receptor specific to the inflammatory cell in the stem cells is increased by the pre-education of stem cells using cell culture medium of the inflammatory cells according to an embodiment of the present invention.

### Example 6: Analysis of Expression Level Through RNA Sequencing in Stem Cells Educated with Inflammatory Synovial Fibroblasts (FLSs) and Inflammatory Macrophages (J774)

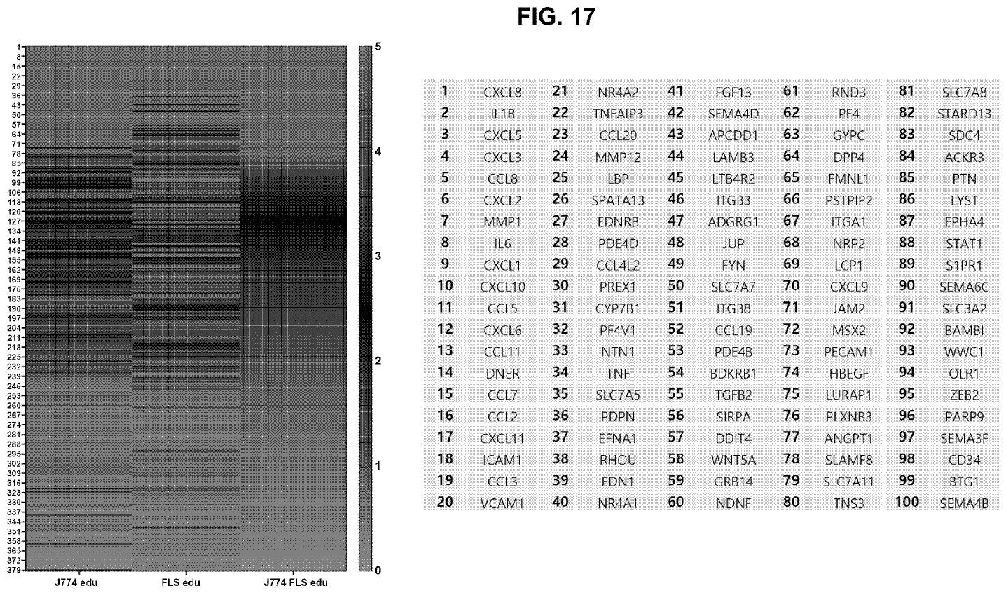

Through RNA sequencing analysis in adipose-derived stem cells educated with inflammatory synovial fibroblasts (FLSs) and inflammatory macrophages (J774A.1), changes in the expression level of genes related to mobility of stem cells were investigated. RNA sequencing analysis was carried out by sequentially proceeding RNA isolation, library preparation and sequencing process, and data analysis process from the educated stem cells. As a result, it was found that the expression of about 100 genes shown in FIG. 17 among the genes related to mobility of adipose-derived stem cells educated with arthritic inflammatory cells according to an embodiment of the present invention increased more than 5 times compared to non-educated stem cells.

RNA Isolation Procedure: Total RNA was isolated using Trizol reagent (Invitrogen). RNA quality was evaluated with an Agilent 2100 bioanalyzer using an RNA 6000 Nano Chip (Agilent Technologies, Amstelveen, Netherlands), and RNA quantification was performed using an ND-2000 Spectrophotometer (Thermo Inc., DE, USA).

Library preparation and sequencing process: For control and test RNA, library preparation was performed using the QuantSeq 3′ mRNA-Seq Library Prep Kit (Lexogen, Inc., Austria) according to the manufacturer's instructions. Briefly, each 500 ng total RNA was prepared and oligo-dT primers containing Illumina compatible sequences at the 5′ end were hybridized to the RNA and reverse transcription was performed. After digestion of the RNA template, second strand synthesis was started by random primers containing an Illumina compatible linker sequence at the 5′ end. The double-stranded library was purified using magnetic beads to remove all reaction components. The library was amplified to add the full adapter sequence required for cluster generation. The finalized library was purified from PCR components. High-throughput sequencing was performed with single-ended 75 sequencing using a NextSeq 500 (Illumina, Inc., USA).

Data analysis process: QuantSeq 3′ mRNA-Seq reads were aligned using Bowtie2 (Langmead and Salzberg, 2012). Bowtie2 indexes were generated from genomic assembly sequences or representative transcript sequences for alignment to the genome and transcriptome. Alignment files were used to assemble transcripts, estimate their amounts, and detect differential expression of genes. Differentially expressed genes were determined based on counts of unique and multiple alignments using the coverage of Bedtools (Quinlan A R, 2010). RC (Read Count) data were processed according to the quantile normalization method using EdgeR in R (R Development Core Team, 2016) using Bioconductor (Gentleman et al., 2004). Genetic classification was performed based on searches performed on DAVID (//david.abcc.ncifcrf.gov/) and Medline database (//www.ncbi.nlm.nih.gov/). Data mining and graphical visualization were performed using ExDEGA (Ebiogen Inc., Korea).

### Example 7: CIA (Collagen-Induced Arthritis) Model Generation

DBA/1 mice (male, 4-6 weeks old, body weight 20-25 g) were purchased from Orient Bio (Seoul, Korea) and the mice were subjected to a 12-hour light/dark cycle (lit at 6:30 am for 7-14 days) and acclimated in a specific pathogen-free environment controlled by temperature and humidity before the experiment. All animal experiments were performed in accordance with the Gachon University Laboratory Animal Care and Use Guide. Arthritis was also induced by intradermal injection of collagen in 2 mg/mL of CFA (Chondrex) via tail vein. Animals with CIA-induced arthritis were randomly assigned to 6 groups (n=6) after the first signs of inflammation were observed on day 27.

### Example 8: Analysis of Arthritis Targeting Ability of Pre-Educated Stem Cells in an Arthritis Model

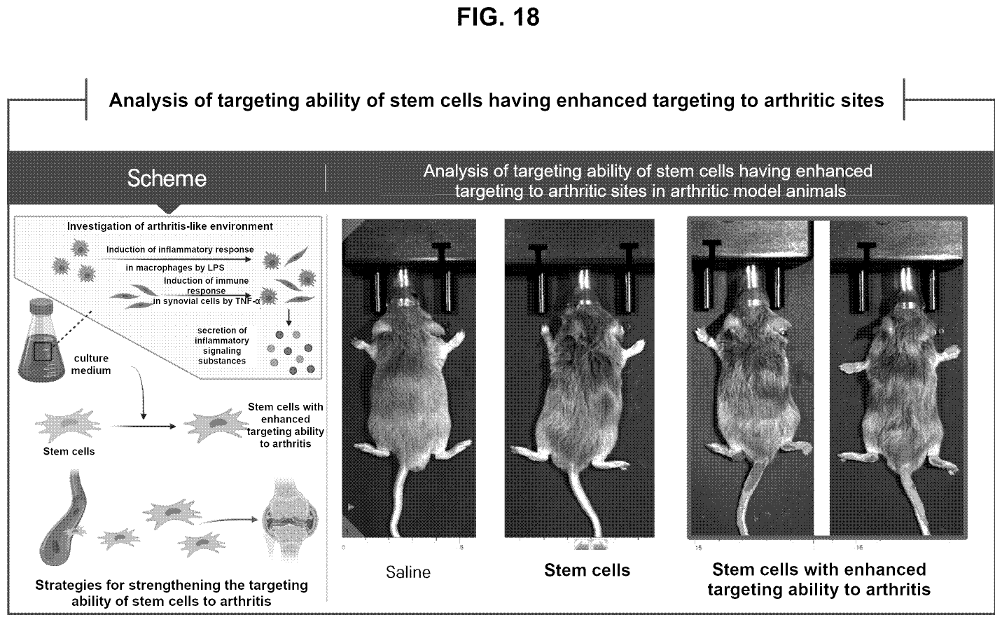

CIA-induced mice were established via intradermal injection of complete Freund's adjuvant (CFA) containing type II collagen to evaluate the arthritic targeting ability of pre-educated AD-MSCs in DBA/1 mice as an arthritis model (FIG. 10A, L. Bevaart, et al., Arthritis Rheum. 66-3362(8): 2192-2205, 2010). The targeting ability of the educated adipose-derived stem cells to the arthritic site was analyzed using an in vivo fluorescence imaging system (IVIS) and histological images. Adipose-derived stem cells (1×10⁶ cells) were each injected intravenously through the tail vein of arthritis model mice once a day for a total of 3 days. The targeting ability of adipose-derived stem cells to the arthritic site was analyzed. As a result, it was confirmed that the adipose-derived stem cells educated with culture medium of the arthritic cells according to an embodiment of the present invention selectively accumulated in the arthritic site (FIG. 18).

Although the present invention has been described with reference to the above-described examples, these are merely exemplary, and those skilled in the art will understand that various modifications and equivalent other embodiments are possible therefrom. Therefore, the true scope of the present invention should be determined by the technical spirit of the appended claims.

What is claimed is:

1. A method for enhancing the ability of stem cells to migrate to an inflammatory site, comprising the step of culturing the stem cells in a culture medium of inflammation-related cells.

2. The method of claim 1, wherein the inflammation-related cells are isolated from the inflammatory site of the patient, or may be the same type of cells stimulated with an inflammation-inducing substance as those of isolated from the inflammatory site of the patient.

3. The method of claim 1, wherein the inflammation-related cells are synovial fibroblasts, inflammation-related macrophages, neutrophils, eosinophils, lymphocytes, or keratinocytes.

4. The method of claim 2, wherein the inflammation-inducing substance is a pro-inflammatory cytokine, an inflammatory chemokine, or a pathogen-derived inflammatory sub stance.

5. The method of claim 4, wherein the inflammatory cytokine is TNF-α, IL-β, IL-8, IL-12, GM-CSF, INF-γ, and IL-18.

6. The method of claim 4, wherein the inflammatory chemokine is MCP-4.

7. The method of claim 4, wherein the pathogen-derived inflammatory substance is an endotoxin or an exotoxin.

8. The method of claim 4, wherein the pathogen-derived inflammatory substance is a toll-like receptor (TLR) ligand.

9. The method of claim 8, wherein the TLR ligand is a lipopolysaccharide (LPS), a triacylated lipoprotein, a diacylated lipoprotein, zymosan, flagellin, dsRNA, ssRNA, or CpG oligodeoxynucleotide (ODN).

10. The method of claim 1, wherein the stem cells are adipose-derived stem cells, umbilical cord blood-derived stem cells, bone marrow-derived stem cells, dental pulp-derived stem cells, muscle-derived stem cells, or dermal stem cells.

11. The method of claim 1, wherein the inflammation is a pathogenic inflammation caused by a pathogen such as bacteria or viruses or an inflammation caused by an autoimmune disease.

12. A pharmaceutical composition for treating an inflammatory disease or an autoimmune disease comprising stem cells having enhanced ability to migrate to an inflammatory site prepared by the method of claim 1 as an active ingredient.

13. The pharmaceutical composition of claim 12, wherein the stem cells having enhanced ability to migrate to an inflammatory site are stem cells in which the expression of ICAM and VCAM is increased 20-fold or more at the mRNA level compared to un-educated stem cells.

14. The pharmaceutical composition of claim 12, wherein the stem cells having enhanced ability to migrate to an inflammatory site are stem cells educated with culture medium of synovial fibroblasts treated with TNF-α, and whose expression level of ICAM is at least 80 times higher, expression level of VCAM is increased at least 100 times higher, and expression level of CXCR4 is at least 10 times higher than those of un-educated stem cells at the mRNA level.

15. The pharmaceutical composition of claim 12, wherein the stem cells having enhanced ability to migrate to an inflammatory site are stem cells educated with culture medium of inflammation-related macrophages or M1 macrophages treated with LPS, and whose expression level of ICAM is increase 20-fold or more compared with un-educated stem cells at the mRNA level.

16. The pharmaceutical composition of claim 12, wherein the stem cells having enhanced ability to migrate to an inflammatory site are stem cells whose expression level of at least one gene selected from the group of consisting of CXCL8, IL1B, CXCL5, CXCL3, CCL8, CXCL2, MMP1, IL6, CXCL1, CXCL10, CCL5, CXCL6 on an mRNA basis, CCL11, DNER, CCL7, CCL2, CXCL11, CXCL11, ICAM1, CCL3, VCAM1, NR4A2, TNFAIP3, CCL20, MMP12, LBP, SPATA13, EDNRB, PDE4D, CCL4L2, S PREX1, CYP7B1, PLCN4V, EFNA1, EFNA1, RHOU, EDN1, NR4A1, FGF1 3, SEMA4D, APCDD1, LAMBS, LTB4R2, ITGB3, ADGRG1, JUP, FYN, SLC7A7, ITGB8, CCL19, PDE4B, BDKRB4, WNT5 ADDIT, PDE4B, BDKITRB4, TGFB2, A GRB14, NDNF, RND3, PF4, GYPC, DPP4, FMNL1, PSTPIP2, ITGA1, NRP2, LCP1, CXCL9, JAM2, MSX2, PECAM1, HBEGF, LURAP1, PLXNB3, ANGPT1, ANGPT1, SLAMF8, SLC7A11, TNSLC3, SLC7A11, SDC4, ACKR3, PTN, LYST, EPHA4, STAT1, S1PR1, SEMA6C, SLC3A2, BAMBI, WWC1, OLR1, ZEB2, PARP9, SEMA3F, SEMA3F, CD34, BTG1, and SEMA4B is increase at least 5-fold compared with un-educated stem cells.

17. The pharmaceutical composition of claim 16, wherein the stem cells having enhanced ability to migrate to an inflammatory site are stem cells educated with culture medium of inflammation-related macrophages or M1 macrophages treated with LPS.

18. The pharmaceutical composition of claim 1, further comprising one or more anti-inflammatory agents.

19. The pharmaceutical composition of claim 18, wherein the anti-inflammatory agent is loaded with a nanoparticle.

20. The pharmaceutical composition of claim 19, wherein the nanoparticle is attached on the surface of the stem cells or loaded inside the stem cells.

21. The pharmaceutical composition of claim 12, wherein the stem cells are adipose-derived stem cells, umbilical cord blood-derived stem cells, bone marrow-derived stem cells, dental pulp-derived stem cells, muscle-derived stem cells, dermal stem cells or induced pluripotent stem cells (iPSCs).

22. The pharmaceutical composition of claim 12, wherein the inflammatory disease is rhinitis, allergic conjunctivitis, epidemic conjunctivitis, hepatitis, bronchitis, laryngitis, tonsillitis, thyroiditis, laryngitis, encephalitis, myelitis, pneumonia, gastritis, colitis, cystitis, pancreatitis, cystitis, synovitis, rheumatoid arthritis, osteoarthritis, ankylosing spondylitis, psoriasis, pruritus, pruritus, seborrheic dermatitis, acne, irritant dermatitis or atopic dermatitis.

23. The pharmaceutical composition, wherein the autoimmune disease is inflammatory bowel disease, rheumatoid arthritis, osteoarthritis, inflammatory myopathy, autoimmune vasculitis, autoimmune hepatitis, autoimmune pancreatitis, autoimmune encephalitis, autoimmune vasculitis, Behcet's disease, systemic lupus, Sjögren's syndrome, myasthenia gravis, scleroderma, polyarteritis nodosa, Kikuchi disease, collagen disease, Hashimoto's thyroiditis, vitiligo, Still's disease, alopecia areata, multiple sclerosis, orthostatic tachycardia syndrome, autoimmune hemolytic anemia, Stevens-Jones syndrome, Galen-Barré syndrome, cytokine storm or pemphigus.

24. The pharmaceutical composition of claim 23, wherein the inflammatory bowel disease is ulcerative colitis or Crohn's disease.

25. The pharmaceutical composition of claim 18, wherein the anti-inflammatory agent is a corticoid-based anti-inflammatory agent or a non-steroid anti-inflammatory agent (NSAID).

26. The pharmaceutical composition of claim 25, wherein the corticoid-based anti-inflammatory agent is hydrocortisone, hydrocortisone acetate, cortisone, cortisone acetate, tixocortol pivalate, hydrocortisone-17-valerate, halometasone, alclometasone dipropionate, betamethasone valerate, betamethasone dipropionate, prednicarbate, clobetasone-17-butyrate, clobetasol-17-propionate, fluocortolone caproate, fluocortolone pivalate, fluprednidene acetate, prednisone, prednisolone, methylprednisolone, dexamethasone, dexamethasone sodium phosphate, betamethasone, betamethasone sodium phosphate, fluocortolone, triamcinolone, triamcinolone acetonide, mometasone, amcinonide, desonide, fluocinonide, fluocinolone acetonide, halcinonide, beclomethasone, fludrocortisone acetate, hydrocortisone-17-butyrate, hydrocortisone aceponate, hydrocortisone ybuteprate, ciclesonide, or prednicarbate.

27. The pharmaceutical composition of claim 25, wherein the non-steroid anti-inflammatory agent may be a cyclooxygenase (COX) inhibitor.

28. The pharmaceutical composition of claim 27, wherein the cyclooxygenase inhibitor is a non-selective COX-1/COX-2 inhibitor, a selective COX-1 inhibitor or a selective COX-2 inhibitor.

29. The pharmaceutical composition of claim 28, wherein the selective COX-2 inhibitor is pricoxib, celecoxib, rofecoxib, parecoxib, lumiracoxib, etoricoxib, or pyrocoxib.

30. The pharmaceutical composition of claim 19, wherein the nanoparticle is gold nanoparticles, carbon nanotubes, liposomes, exosomes, or nanoparticles having a core/shell structure including a biodegradable polymer.

31. The pharmaceutical composition of claim 30, wherein the nanoparticle is attached on the surface of the stem cells via an antibody specific for a stem cell-specific surface marker, which is attached to the surface of the nanoparticle.
